(19) United States
(12) Patent Application Publication
(10) Pub. No.: US 2013/0059349 A1
(43) Pub. Date: Mar. 7, 2013

(54) METHOD FOR PRODUCING ACRYLAMIDE USING MICROBIAL CATALYST

(71) Applicants: Kozo Murao, Yokohama-shi (JP); Makoto Kano, Yokohama-shi (JP); Yuji Hirata, Yokohama-shi (JP)

(72) Inventors: Kozo Murao, Yokohama-shi (JP); Makoto Kano, Yokohama-shi (JP); Yuji Hirata, Yokohama-shi (JP)

(73) Assignee: DIA-NITRIX CO., LTD., Tokyo (JP)

(21) Appl. No.: 13/696,193

(22) PCT Filed: May 6, 2011
(86) PCT No.: PCT/JP2011/060606

§ 371 Date: Nov. 5, 2012

(30) Foreign Application Priority Data
May 6, 2010 (JP) 2010-106562

(51) Int. Cl. C12P 13/02; C12M 1/38

(52) U.S. Cl. 435/129; 435/286.1

(57) ABSTRACT

The present invention provides a more efficient method for producing acrylamide from acrylonitrile by the action of a microbially-derived enzyme, nitrile hydratase. More specifically, the present invention provides a method for producing acrylamide from acrylonitrile using a biocatalyst having nitrile hydratase, which comprises the step of keeping acrylonitrile while cooling to less than 30° C. Moreover, the present invention also provides an apparatus for producing acrylamide from acrylonitrile using a biocatalyst having nitrile hydratase.

### TECHNICAL FIELD

The present invention relates to a method for producing acrylamide from acrylonitrile by the action of a microbially-derived enzyme, nitrile hydratase. More specifically, the present invention relates to a method and apparatus for producing acrylamide from acrylonitrile kept at a temperature of less than 30° C. by the action of nitrile hydratase.

### BACKGROUND ART

Acrylamide is used as an industrially important substance in a wide range of fields. For example, polymers of acrylamide are widely used in flocculating agents for waste water treatment, paper strength enhancers, petroleum recovering agents, etc. Industrial production of acrylamide has conventionally been accomplished by hydration of the corresponding acrylonitrile using copper in a reduced state as a catalyst. In recent years, techniques using biocatalysts (microbial catalysts) instead of copper catalysts have been developed, some of which have been in practical use. Biocatalyst-mediated techniques are promising candidates for industrial production because they require mild reaction conditions, produce almost no by-products and ensure a very simple process. Until now, there have been found may microorganisms containing the enzyme nitrile hydratase that has a catalytic ability to convert acrylonitrile into acrylamide through hydration.

A method for producing acrylamide using a microbial catalyst includes the methods described in Patent Documents 1 to 3, and a procedure for reaction includes that described in Patent Document 4 and so on.

Many studies have also been conducted on efficient procedures for reaction (Patent Documents 5 to 9).

Moreover, to produce high performance acrylamide in a more efficient manner, various studies have been made to treat acrylonitrile or to use acrylonitrile with fewer impurities (Patent Documents 10 to 15).

However, as to the temperature at which acrylonitrile is preserved or stored, standard MSDS (Material Safety Data Sheet) documents or the like state that acrylonitrile should be kept in a cool dark place (e.g., Non-patent Document 1), although no study has been conducted to examine the effect of the preservation temperature for acrylonitrile on hydration reaction of acrylamide.

### PRIOR ART DOCUMENT(S)

### Patent Document(s)

Patent Document 1: JP H11-123098 A
Patent Document 2: JP H7-265091 A
Patent Document 3: JP S56-38118 B (Kokoku Publication)
Patent Document 4: JP H11-89575 A
Patent Document 5: JP 2004-524047 A
Patent Document 6: CN 1482250 A
Patent Document 7: WO2007/097292
Patent Document 8: WO2007/132601
Patent Document 9: WO03/000914
Patent Document 10: JP H9-227478 A
Patent Document 11: JP 2000-016978 A
Patent Document 12: JP H11-123098 A
Patent Document 13: JP 2001-288156 A
Patent Document 14: WO2007/043466
Patent Document 15: WO2004/090148

### Non-Patent Document(s)

Non-patent Document 1: Product Safety Data Sheet for Acrylonitrile, prepared by Dia-Nitrix Co., Ltd., Japan

### SUMMARY OF THE INVENTION

### Problem to be Solved by the Invention

The object of the present invention is to provide a method and apparatus for producing acrylamide at a high concentration.

### Means to Solve the Problem

As a result of extensive and intensive efforts made to solve the problems stated above, as to the relationship between the preservation temperature for acrylonitrile and the efficiency of acrylamide-producing reaction using the acrylonitrile as a raw material, the inventors of the present invention have found that the use of acrylonitrile kept at a temperature of less than 30° C. allows production of acrylamide at a higher concentration with a smaller amount of catalyst. This finding led to the completion of the present invention.

Namely, the present invention is as follows.

(1) A method for producing acrylamide from acrylonitrile using a biocatalyst having nitrile hydratase,

which comprises the step of keeping acrylonitrile while cooling to less than 30° C.

(2) An apparatus for producing acrylamide from acrylonitrile using a biocatalyst having nitrile hydratase, which comprises a temperature regulation mechanism for maintaining the temperature of acrylonitrile at less than 30° C.

### Effects of the Invention

When using acrylonitrile preserved at less than 30° C., high quality acrylamide can be produced at a higher concentration with a smaller amount of catalyst. Namely, the production method of the present invention significantly increases the amount of compound produced per unit amount of catalyst (i.e., the production efficiency of the catalyst (hereinafter also simply referred to as “productivity”)), as compared to conventional production methods.

Moreover, it is possible to reduce various organic impurities (saccharides or proteins) and/or inorganic impurities (minerals), which are brought in or extracted from biocatalysts or suspensions thereof, thus resulting in acrylamide of higher purity.

### BRIEF DESCRIPTION OF THE DRAWINGS

FIG. 1 is a perspective view illustrating a storage apparatus which comprises an acrylonitrile cooling mechanism for use in the acrylamide production apparatus of the present invention.

FIG. 2 is an explanation drawing illustrating an outline of an acrylamide production apparatus which comprises an acrylonitrile storage apparatus.

### EMBODIMENTS FOR CARRYING OUT THE INVENTION

Hereinafter, embodiments of the present invention will be described. The following embodiments are examples provided for illustrating the present invention, and the present invention is not intended to be limited thereto. The present invention may be carried out in various embodiments without departing from the spirit of the invention.

The present specification incorporates the content of the specification of Japanese Patent Application No. 2010-106562 (filed on May 6, 2010) based on which the present application claims priority. All publications cited herein, including technical literatures, patent laid-open publications, patent publications and other patent documents, are incorporated herein by reference in their entirety.

A method for acrylamide production using a biocatalyst may be accomplished by continuous reaction (acrylamide is produced in a continuous manner) or by batch reaction (acrylamide is produced in a non-continuous manner). Preferred is, but not limited to, the method accomplished by continuous reaction.

As used herein, a method accomplished by continuous reaction is intended to mean a method wherein acrylamide is produced in a continuous manner without collecting the entire reaction mixture in the reactor while maintaining continuous or intermittent supply of raw materials for reaction (comprising a biocatalyst and acrylonitrile) and continuous or intermittent recovery of the reaction mixture (comprising the produced acrylamide).

Although the acrylonitrile concentration during reaction will vary depending on the type and/or form of biocatalyst to be used, it is preferably around 0.5% to 15.0% by weight.

When the production method of the present invention is accomplished by continuous reaction, the flow rate upon collection of the reaction mixture from the reactor may be determined in line with the introduction rate of acrylonitrile and the biocatalyst so as to ensure continuous production without collecting the entire reaction mixture in the reactor.

The biocatalyst to be used in the present invention includes animal cells, plant cells, cell organelles, microbial cells (living or dead microbial cells) or treated products thereof, which contain an enzyme (i.e., nitrile hydratase) catalyzing a desired reaction. Such treated products include a crude or purified enzyme extracted from the cells, as well as animal cells, plant cells, cell organelles, microbial cells (living or dead microbial cells) or enzyme molecules which are immobilized by entrapping, crosslinking or carrier binding techniques, etc. Preferably, a biocatalyst having nitrile hydratase is intended to mean microbial cells containing an enzyme having nitrile hydratase activity or treated products thereof, or alternatively, microbial cells or enzyme molecules which are immobilized by entrapping, crosslinking or carrier binding techniques, etc. Entrapping includes a technique by which microbial cells or enzymes are enclosed within a fine lattice of polymer gel or coated with a semipermeable polymer membrane. Crosslinking includes a technique by which enzymes are crosslinked with a reagent having two or more functional groups (i.e., a multifunctional crosslinking agent). Carrier binding includes a technique by which enzymes are bound to a water-insoluble carrier. Examples of a carrier for immobilization include glass beads, silica gel, polyurethane, polyacrylamide, polyvinyl alcohol, carrageenan, alginic acid, agar, gelatin, etc.

Among techniques used for immobilization of microbial cells, entrapping immobilization is often used for industrial purposes because it is possible to obtain immobilized microbial cells with a high microbial cell concentration. For example, cases where acrylamide and/or an acrylamide derivative is used as a monomer for entrapping immobilization can be found in JP S58-35078 B (Kokoku Publication) and JP H7-203964 A.

Preferably, the microorganism having nitrile hydratase activity according to the present invention includes, but are not limited to, a microorganism belonging to the genera Bacillus, Bacteridium, Micrococcus, Brevibacterium [JP S62-21519 B (Kokoku Publication)], Corynebacterium, Nocardia [JP S56-17918 B (Kokoku Publication)], Pseudomonas [JP S59-37951 B (Kokoku Publication)], Microbacterium [JP H4-4873 B (Kokoku Publication)], Rhodococcus [JP H4-4873 B (Kokoku Publication), JP H6-55148 B (Kokoku Publication), JP H7-40948 B (Kokoku Publication)], Achromobacter [JP H6-225780 A] and Pseudonocardia [JP H9-275978 A]. More preferred are bacteria of the genus Rhodococcus. Even more preferred microbial cells include Rhodococcus rhodochrous strain J1 (FERM BP-1478).

Rhodococcus rhodochrous strain J1 having nitrile hydratase activity was internationally deposited on Sep. 18, 1987 under Accession No. FERM BP-1478 with the International Patent Organism Depositary, the National Institute of Advanced Industrial Science and Technology (Chuo 6, 1-1-1 Higashi, Tsukuba, Ibaraki, Japan).

Information about the depositor is as follows.
Name: Hideaki Yamada
Address: 19-1 Kinomoto-cho, Matsugasaki, Sakyo-ku, Kyoto-shi, Kyoto

Alternatively, nitrile hydratase genes derived from the above microorganisms may be obtained and introduced into any hosts, either directly or after being artificially modified, and the resulting transformants may be used.

These transformants may be exemplified by E. coli MT10770 transformed with nitrile hydratase of the genus Achromobacter (FERM P-14756) (JP H8-266277 A), E. coli MT10822 transformed with nitrile hydratase of the genus Pseudonocardia (FERM BP-5785) (JP H9-275978 A) or microorganisms transformed with nitrile hydratase of the species Rhodococcus rhodochrous (JP H4-211379 A). Moreover, desired transformants may also be prepared in accordance with the procedures described in the above documents or other known procedures (Molecular Cloning, A Laboratory Manual 2nd ed., (Cold Spring Harbor Laboratory Press (1989), Current Protocols in Molecular Biology, (John Wiley & Sons (1987-1997)). In the method of the present invention, any transformant may be used as a biocatalyst as long as it expresses the nitrile hydratase gene.

Although the amount of the biocatalyst to be used will vary depending on the type and/or form of the biocatalyst, it is preferably adjusted such that the activity of the biocatalyst to be introduced into a reactor is around 50 to 200 U per mg of dried microbial cells at a reaction temperature of 10° C. The above unit “U (unit)” is intended to mean that one micromole of acrylamide is produced for one minute from acrylonitrile, which is measured by using acrylonitrile to be used for production. When compared to the amount of the biocatalyst used in acrylamide production using acrylonitrile kept at 30° C. or higher, the production method of the present invention allows reaction with a smaller amount of the biocatalyst or allows acrylamide production in higher yields with the same amount of the biocatalyst.

As used herein, keeping of the raw material acrylonitrile is intended to mean, for example, that acrylonitrile is kept for a given period of time or longer (e.g., 1 day or longer, preferably 3 days or longer, more preferably 7 days or longer) in a keeping/storage equipment for acrylamide included in an acrylamide production equipment. Examples of such a keeping/storage equipment include an equipment for keeping a drum containing acrylonitrile and a tank for storing acrylonitrile, etc. For industrial production of acrylamide, an acrylonitrile storage tank is generally preferred.

The storage equipment for acrylonitrile is preferred to have light-shielding properties, oxygen insulation properties, firesafe properties, antistatic properties, antivibration properties and so on, and is further desired to have a mechanism capable of tightly closing the storage environment and optionally allowing exhaustion and ventilation. There is no particular limitation for a storage form of acrylonitrile as long as its stability is ensured, although stabilizers may optionally be added to increase the stability of acrylonitrile.

Keeping acrylonitrile while cooling to less than 30° C. is intended to mean that acrylonitrile is kept while being cooled such that the internal acrylonitrile temperature does not become 30° C. or more during storage over the summer months or in high temperature areas.

The present invention also provides an acrylamide production apparatus (equipment) which comprises a cooling apparatus for keeping acrylonitrile while cooling. There is no particular limitation for a cooling apparatus used to prevent elevation of the internal temperature, as long as acrylonitrile can be cooled by means of a cooling fluid. Although there is no particular limitation for the cooling fluid, a liquid whose heat capacity is greater than that of gas allows more efficient cooling of acrylonitrile.

Among cooling liquids, water is particularly preferred for use because of its low cost and easy handling.

In such a cooling equipment with a tank as described above, acrylonitrile can be cooled in various manners. Examples of a cooling mechanism for use in the storage tank for acrylonitrile include a mechanism by which water or cooling water is sprayed over the tank surface, a jacket mechanism for cooling which is provided on the tank wall, or a mechanism by which a cooling solution (coolant) such as cooling water or an aqueous ethylene glycol solution is passed through a coil provided on the tank wall or in the tank interior. The cooling water or coolant may be repeatedly used by being circulated. However, when water is sprayed, it can be drained without being circulated for repeated use because it is inexpensive and free from any environmental load.

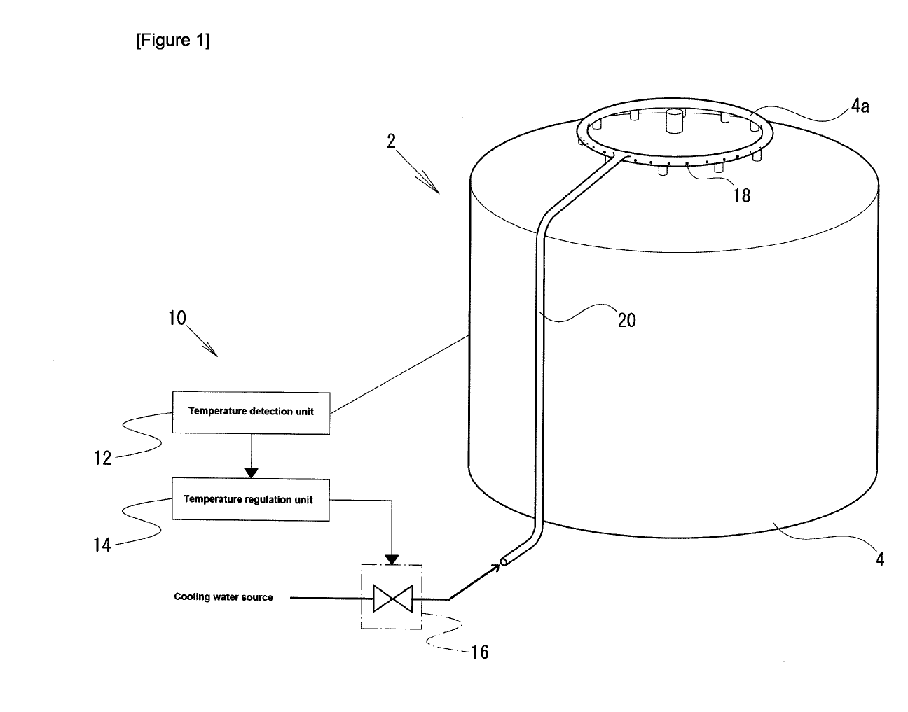

By reference to FIG. 1, a more detailed explanation will be given below of a storage apparatus for acrylonitrile comprising a water spray equipment. The storage apparatus for acrylonitrile as shown in FIG. 1 is merely an illustrative example and how to cool acrylonitrile is not limited to the following explanation.

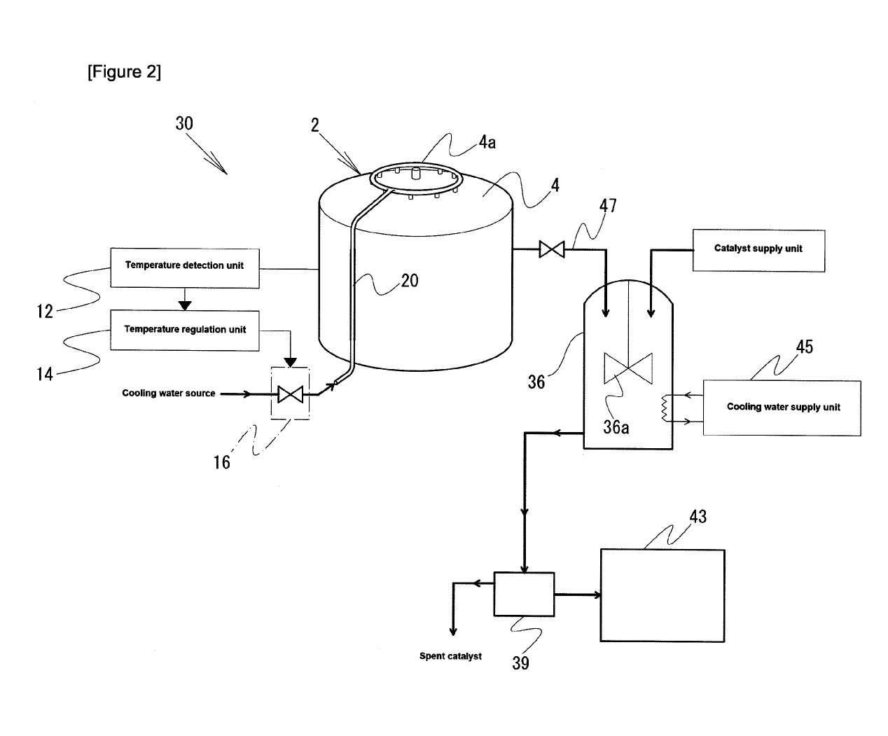

As shown in FIG. 1, a storage apparatus for acrylonitrile 2 is configured such that it can be included in an acrylamide production apparatus 30 (see FIG. 2), and it comprises a storage tank 4 in which acrylonitrile is stored, and a cooling mechanism 10 by which acrylonitrile accommodated in the tank 4 is cooled to, e.g., less than 30° C.

The storage tank 4 is formed from a material with high thermal conductivity (e.g., a metal), and the tank surface is treated for corrosion resistance so as to prevent corrosion caused by water, etc.

Although the cooling mechanism for use in the storage tank 4 may be of inner coil type or jacket type, the cooling mechanism is preferably of spray type in terms of low running costs, etc. Such a spray-type cooling mechanism 10 comprises a temperature detection unit 12, a cooling water valve 16 and so on. If the temperature of acrylonitrile is empirically estimated from climatic conditions including outside air temperature and weather patterns, the temperature detection unit 12 may not be used.

In the case of using the cooling mechanism 10 with the temperature detection unit 12, the temperature of acrylonitrile is controlled by feedback based on the temperature information inputted from the temperature detection unit 12, the opening and closing of the cooling water valve 16 is controlled by a temperature regulation unit 14 on the basis of the temperature information to thereby regulate the temperature of acrylonitrile. Cooling water supplied from a cooling water source may be, e.g., cooled water or an aqueous ethylene glycol solution, which is adjusted to a temperature of 5° C. to 20° C. Alternatively, if industrial water or the like can be obtained at low cost, industrial water may be used directly. In the case of using industrial water, the sprayed water can be drained without being recovered.

On the top of the storage tank 4, a water spray ring 4a which is formed into, e.g., a ring shape is provided in order to spray cooling water, and cooling water is supplied from the cooling water source through a cooling water supply pipe 20 into the water spray ring 4a. The water spray ring has small water spray holes 18 provided on its outer circumference to ensure that the surface of the storage tank 4 can be uniformly wetted with water. The cooling water sprayed from the water spray ring 4a through the water spray holes 18 cools the surface of the storage tank 4, e.g., by flowing down on the tank surface, whereby acrylonitrile in the storage tank 4 is cooled. The water spray ring 4a and the water spray holes 18 may be of any size, without particular limitation, as long as the storage tank 4 can be cooled. For example, the water spray ring 4a may be designed to have a diameter of 40 mm, and water spray holes 18 may be provided with a diameter of 3.5 mm and at 75 mm intervals on this water spray ring 4a.

To drive the cooling water valve 16, for example, the cooling water valve 16 may be opened to supply cooling water in the direction toward the storage tank 4 if the temperature of acrylonitrile detected by the temperature detection unit 12 exceeds a threshold value (e.g., 25° C.), whereas the cooling water valve 16 may be closed if the detected acrylonitrile temperature is equal to or less than the threshold value.

By opening the cooling water valve 16 to supply cooling water in the direction toward the storage tank 4 in this way, acrylonitrile stored within the storage tank 4 can be maintained at less than 30° C.

Next, further explanation will be given below of an acrylamide production apparatus comprising a storage apparatus for acrylonitrile by reference to FIG. 2. As to the storage apparatus for acrylonitrile, the same elements as found in the storage apparatus illustrated in FIG. 1 are indicated with the same reference numerals for brief explanation, and their detailed explanation is omitted.

As shown in FIG. 2, an acrylamide production apparatus 30 comprises a storage apparatus for acrylonitrile 2, a reactor 36, a separator 39, an acrylamide reservoir tank 43, a cooling water supply unit 45 and so on.

The cooling water supply unit 45 supplies cooling water to the reactor 36 through a cooling water path, so that the reactor 36 can be cooled by the supplied cooling water. The cooling water flowing out of the reactor 36 is returned to the cooling water supply unit 45 through the cooling water path.

In the production apparatus 30, acrylonitrile is supplied from the storage tank for acrylonitrile 2 to the reactor 36 through a supply line 47, and the acrylonitrile supplied to the reactor 36 is mixed with a biocatalyst by means of, e.g., a stirring blade 36a to produce acrylamide through nitrile hydratase reaction. The reaction mixture containing acrylamide is discharged from the reactor 36, and the discharged reaction mixture is supplied to the separator 39, as exemplified by a centrifugal separator, etc.

Acrylamide is separated from the reaction mixture supplied to the separator 39. The separated acrylamide is held in the acrylamide reservoir tank 43, while the separated biocatalyst is disposed as spent catalyst.

The temperature of acrylonitrile in the tank 4 is controlled, e.g., to be equal to or less than 25° C., and acrylonitrile thus controlled is supplied to the reactor 36 to thereby achieve not only efficient production of acrylamide, but also provision of high quality acrylamide.

Although a temperature of 25° C. was exemplified as a threshold for cooling in the above explanation, the threshold is not limited only to this temperature and may be altered as appropriate, depending on the nature of the biocatalyst used for nitrile hydratase reaction and/or the temperature during the reaction. In particular, the activity level of microbial nitrile hydratase may vary depending on various conditions. In such a case, it is desired that the reaction temperature is determined on the basis of the conditions used, while the threshold settings for acrylonitrile cooling are altered flexibly.

As described above, in the acrylamide production apparatus of the present invention, acrylonitrile can be maintained at a temperature of less than 30° C. without being affected by the temperature of the external environment (ambient temperature) where this apparatus is placed. For example, in the case of a production apparatus comprising a cooling mechanism in which cooling water is used, if the ambient temperature of this apparatus is higher than the temperature preferred for acrylamide production, the cooling water may be used more positively. In this case, it is preferable to monitor the temperature of acrylonitrile, although the cooling mechanism may be driven by judging from air temperature and/or sunlight intensity without monitoring the temperature of acrylonitrile. Alternatively, instead of using a valve or pump which controls cooling water supply, cooling water at a constant temperature less than 30° C. may be continuously circulated in the storage tank for acrylonitrile to thereby maintain acrylonitrile at a temperature of less than 30° C. Such a configuration also allows efficient production of acrylamide.

### EXAMPLES

The present invention will be further described in more detail by way of the following examples, which are not intended to limit the scope of the present invention.

### Examples 1 and 2

### Production of Acrylamide Using Acrylonitrile Kept at 20° C. or 28° C.

(Keeping of Acrylonitrile)

Acrylonitrile (Dia-Nitrix Co., Ltd., Japan) was introduced into a 500 mL glass bottle and kept for 7 days in a thermostatic chamber adjusted to 20° C. or 28° C.

(Preparation of Biocatalyst)

Rhodococcus rhodochrous J1 having nitrile hydratase activity (FERM BP-1478) was cultured at 30° C. under aerobic conditions in a medium (pH 7.0) containing 2% glucose, 1% urea, 0.5% peptone, 0.3% yeast extract and 0.05% cobalt chloride (all in % by weight). This culture was washed with 50 mM phosphate buffer (pH 7.0) using a centrifugal separator to obtain a microbial cell suspension (dried microbial cells: 15% by weight).

(Reaction Converting Acrylonitrile into Acrylamide)

A 1 L jacketed separable flask was charged with deionized water (664 g), and the water temperature was controlled at 18° C. After 30 minutes, the microbial cell suspension obtained above (0.8 g) was added and acrylonitrile was continuously added thereto under stirring at 180 rpm, such that the acrylonitrile concentration was kept constant at 2%, to thereby initiate production of acrylamide.

As a result, in either case of using acrylonitrile kept at a temperature of 20° C. or 28° C., the concentration of acrylamide produced within 25 hours from the initiation of acrylonitrile addition was found to reach a desired level of 45%.

### Comparative Example 1

Except for using acrylonitrile kept at 35° C., the same procedure as shown in Example 1 was performed, indicating that the acrylamide concentration was only 42% within 25 hours. For this reason, the amount of the microbial cells to be added was increased to 0.9 g. As a result, the acrylamide concentration was found to reach 45% within 25 hours.

The above results indicated that the use of acrylonitrile kept at a temperature of less than 30° C. allowed production of acrylamide with a smaller amount of biocatalyst, when compared to the use of acrylonitrile kept at 30° C. or higher.

### Example 3

### Preparation of Transformant Having Nitrile Hydratase Derived from Rhodococcus rhodochrous Strain M8

(1) Preparation of Chromosomal DNA from Rhodococcus rhodochrous Strain M8 (Hereinafter Referred to as the Strain M8)

The strain M8 (SU1731814) is available from the Institute of Biochemistry and Physiology of Microorganisms (IBFM) in Russia (VKPM S-926).

The strain M8 was cultured under shaking at 30° C. for 72 hours in 100 ml of MYK medium (pH 7.0; 0.5% polypeptone, 0.3% Bactoyeast extract, 0.3% Bactomalt extract, 0.2% K₂HPO₄, 0.2% KH₂PO₄). The cultured solution was centrifuged and the collected microbial cells were suspended in 4 ml of a Saline-EDTA solution (0.1 M EDTA, 0.15 M NaCl (pH 8.0)). To this suspension, lysozyme (8 mg) was added and shaken at 37° C. for 1 to 2 hours, followed by freezing at −20° C.

Then, to this suspension, 10 ml of a Tris-SDS solution (1% SDS, 0.1 M NaCl, 0.1 M Tris-HCl (pH 9.0)) was added under mild shaking. Further, to this suspension, proteinase K (Merck & Co., Inc.) (final concentration: 0.1 mg) was added and shaken at 37° C. for 1 hour. Then, an equal volume of TE-saturated phenol is added and stirred (TE: 10 mM Tris-HCl, 1 mM EDTA (pH 8.0)), followed by centrifugation. The upper layer was collected and supplemented with two volumes of ethanol, and DNA was wound around a glass bar. This was then centrifuged sequentially with 90%, 80% and 70% ethanol to remove phenol.

Then, the DNA was dissolved in 3 ml of TE buffer, to which a ribonuclease A solution (treated by heating at 100° C. for 15 minutes) was then added to be at a concentration of 10 μg/ml and shaken at 37° C. for 30 minutes. Further, proteinase K (Merck & Co., Inc.) was added and shaken at 37° C. for 30 minutes. This mixture was supplemented with an equal volume of TE-saturated phenol and centrifuged to separate into upper and lower layers.

The upper layer was further supplemented with an equal volume of TE-saturated phenol and centrifuged to separate into upper and lower layers. This procedure was repeated again. Then, the upper layer was supplemented with an equal volume of chloroform (containing 4% isoamyl alcohol) and centrifuged to collect the upper layer. Then, the upper layer was supplemented with two volumes of ethanol, and DNA was collected by being wound around a glass bar to obtain chromosomal DNA.

(2) Preparation of Nitrile Hydratase Gene from Chromosomal DNA of Strain M8 Using PCR

Nitrile hydratase derived from the strain M8 can be found in Veiko, V. P. et al., Cloning, nucleotide sequence of nitrile hydratase gene from Rhodococcus rhodochrous M8, Biotekhnologiia (Mosc.) 5, 3-5 (1995), and the sequences of its β-subunit, α-subunit and activator are shown in Table 1.

TABLE 1

| Strain M8 | Base sequence | Amino acid sequence |
|---|---|---|
| β-subunit | SEQ ID NO: 1 | SEQ ID NO: 2 |
| α-subunit | SEQ ID NO: 3 | SEQ ID NO: 4 |
| Activator | SEQ ID NO: 5 | SEQ ID NO: 6 |

Based on the above sequence information, primers M8-1 and M8-2 were synthesized and PCR was performed using the chromosomal DNA prepared in (1) as a template.

### <Primers>

(SEQ ID NO: 7)
M8-1: GGTCTAGAATGGATGGTATCCACGACACAGGC
(SEQ ID NO: 8)
M8-2: CCCCTGCAGGTCAGTCGATGATGGCCATCGATTC

### <Composition of PCR Reaction Solution>

Template DNA (chromosomal DNA) 200 ng

PrimeSTAR Max Premix (TaKaRa Shuzo Co., Ltd., Japan) 25 μl

Primer M8-1 10 pmol

Primer M8-2 10 pmol

### <Reaction Conditions>

(98° C. for 10 seconds, 55° C. for 5 seconds, and 72° C. for 30 seconds)×30 cycles

After completion of the PCR, the reaction solution (5 μl) was subjected to electrophoresis on a 0.7% agarose gel (using Agarose I, a product of Dojindo Laboratories, Japan; agarose concentration: 0.7% by weight) to detect a 1.6 kb amplified fragment. The reacted solution was purified with a Wizard SV Gel and PCR Clean-Up System (Promega KK).

The collected PCR product was ligated to a vector (pUC118/HincII site) using a Ligation Kit (TaKaRa Shuzo Co., Ltd., Japan), and the reaction solution was used to transform E. coli JM109 competent cells. Some clones from the resulting transformant colony were inoculated into LB-Amp medium (1.5 ml) and cultured under shaking at 37° C. for 12 hours. After culturing, this cultured product was centrifuged to collect the microbial cells. A QIAprep Spin Miniprep Kit (Amersham Biosciences) was used to extract the plasmid DNA from the collected microbial cells. The resulting plasmid DNA was subjected to a sequencing kit and an autosequencer CEQ 8000 (Beckman Coulter) to confirm the base sequence of nitrile hydratase.

Then, the resulting plasmid DNA was digested with restriction enzymes XbaI and Sse8387I, and then electrophoresed on a 0.7% agarose gel to collect a nitrile hydratase gene fragment (1.6 kb), which was then introduced into a XbaI-Sse8387I site in plasmid pSJ042. The resulting plasmid was designated as pSJ-N01A.

pSJ042 was prepared as described in JP 2008-154552 A as a plasmid expressing the strain J1 nitrile hydratase in Rhodococcus spp., and pSJ023 used for preparation of pSJ042 was deposited as the transformant ATCC12674/pSJ023 (FERM BP-6232) on Mar. 4, 1997 with the International Patent Organism Depositary, the National Institute of Advanced Industrial Science and Technology (Chuo 6, 1-1-1 Higashi, Tsukuba, Ibaraki, Japan).

Information about the depositor is as follows.
Name: Mitsubishi Rayon Co., Ltd.
Address: 6-41, Konan 1-chome, Minato-ku, Tokyo

(3) Preparation of Competent Cells

Rhodococcus rhodochrous strain ATCC 12674 (hereinafter referred to as the strain ATCC 12674) was cultured in MYK medium until the early stage of the logarithmic growth phase, and the cells were collected with a centrifugal separator, washed three times with ice-cold sterilized water and then suspended in sterilized water to prepare competent cells.

(4) Preparation of Transformant Having Nitrile Hydratase Derived from Strain M8

The resulting plasmid pSJ-N01A (0.1 μg) and a microbial cell suspension of the competent cells from the strain ATCC 12674 (20 μl each) were mixed together and cooled on ice. Each mixture was introduced into a cuvette and electrically pulsed in a gene transfer device, Gene Pulser (BIO RAD), at 20 KV/cm at 200 OHMS. The electrically pulsed solution was allowed to stand under ice cooling for 10 minutes and heat shocked at 37° C. for 10 minutes. Then, the cuvette was supplemented with MYK medium (500 μl) and allowed to stand at 30° C. for 5 hours, and then applied onto 50 μg/ml kanamycin-containing MYK agar medium and cultured at 30° C. for 3 days.

The plasmid DNA contained in the resulting transformant colony was confirmed, and this recombinant strain was defined as a recombinant Rhodococcus sp. strain (ATCC12674/pSJ-N01A) having nitrile hydratase derived from the strain M8.

(5) Adjustment of Recombinant Rhodococcus sp. Strain

This strain was cultured in the same manner as shown in Example 1 to obtain a recombinant microbial cell suspension (dried microbial cells: 6% by weight).

(6) Reaction Converting Acrylonitrile into Acrylamide by the Recombinant Strain

A 1 L jacketed separable flask was charged with deionized water (600 g), and the water temperature was controlled at 25° C. After 30 minutes, the recombinant microbial cell (ATCC12674/pSJ-N01A) suspension obtained above (5 g) was added and acrylonitrile which had been kept at room temperature (25° C. or lower) was continuously added thereto at an addition rate of 84 g/h under stirring at 180 rpm to thereby initiate production of acrylamide.

After 4 hours, the acrylamide concentration was found to reach a desired level of 40%. The productivity of this reaction was about 900.

### Comparative Example 2

Except for using acrylonitrile kept at 35° C., the same procedure as shown in Example 2 was performed, indicating that the acrylamide concentration was 38% after 4 hours and did not reach a desired level of 40% over the subsequent hours with increases being observed only in the acrylonitrile concentration.

### Example 4

### Preparation of Transformant Having Nitrile Hydratase Derived from Pseudonocardia thermophila Strain JCM3095

(1) Preparation of Nitrile Hydratase Gene from pPt-DB1 Plasmid DNA Using PCR

pPT-DB 1 is a plasmid containing the nitrile hydratase gene derived from Pseudonocardia thermophila strain JCM3095 (hereinafter referred to as the strain JCM3095) obtained in JP H9-275978 A.

The strain JCM3095 can be found in JP H9-275978 A, and the sequences of its β-subunit, α-subunit and activator are shown in Table 2.

TABLE 2

| Strain JCM3095 | Base sequence | Amino acid sequence |
|---|---|---|
| β-subunit | SEQ ID NO: 9 | SEQ ID NO: 10 |
| α-subunit | SEQ ID NO: 11 | SEQ ID NO: 12 |
| Activator | SEQ ID NO: 13 | SEQ ID NO: 14 |

Based on the above sequence information, primers PSN-1 and PSN-1 were synthesized and PCR was performed using the pPT-DB1 plasmid DNA as a template.

### <Primers>

(SEQ ID NO: 15)
PSN-1: GGTCTAGAATGAACGGCGTGTACGACGTCGGC
(SEQ ID NO: 16)
PSN-2: ccCCTGCAGGTCAGGACCGCACGGCCGGGTGGAC

### <Composition of PCR Reaction Solution>

Template DNA (pPT-DB 1) 200 ng

PrimeSTAR Max Premix (TaKaRa Shuzo Co., Ltd., Japan) 25 μl

Primer PSN-1 10 pmol

Primer PSN-2 10 pmol

### <Reaction Conditions>

(98° C. for 10 seconds, 55° C. for 5 seconds, and 72° C. for 30 seconds)×30 cycles

The resulting PCR product was treated in the same manner as shown in Example 1(2) to prepare a plasmid, which was designated as pSJ-N02A.

(2) Preparation of Transformant Having Nitrile Hydratase Derived from Strain JCM3095

The same procedure as shown in Example (4) was repeated to prepare a recombinant Rhodococcus sp. strain (ATCC12674/pSJ-N02A) having nitrile hydratase derived from the strain JCM3095.

(3) Adjustment of Recombinant Rhodococcus sp. Strain

This strain was cultured in the same manner as shown in Example 1 to obtain a recombinant microbial cell suspension (dried microbial cells: 5% by weight).

(4) Reaction Converting Acrylonitrile into Acrylamide by Recombinant Strain

A 1 L jacketed separable flask was charged with deionized water (700 g), and the water temperature was controlled at 25° C. After 30 minutes, the microbial cell suspension obtained above (12 g) was added and acrylonitrile which had been kept at room temperature (25° C. or lower) was continuously added thereto at an addition rate of 84 g/h under stirring at 180 rpm to thereby initiate production of acrylamide.

After 2 hours, the acrylamide concentration was found to reach a desired level of 20%.

### Comparative Example 3

Except for using acrylonitrile kept at 35° C., the same procedure as shown in Example 5 was performed, indicating that the acrylamide concentration was 18% after 2 hours and did not reach a desired level of 20% over the subsequent hours with increases being observed only in the acrylonitrile concentration.

The above results indicated that even in the case of using a transformant as a biocatalyst, the use of acrylonitrile kept at a temperature of less than 30° C. also allowed efficient production of acrylamide when compared to the use of acrylonitrile kept at 30° C. or higher.

### INDUSTRIAL APPLICABILITY

The method of the present invention enables more efficient production of acrylamide.

### SEQUENCE LISTING FREE TEXT

SEQ ID NO: 7: synthetic DNA
SEQ ID NO: 8: synthetic DNA
SEQ ID NO: 15: synthetic DNA
SEQ ID NO: 16: synthetic DNA

1. A method for producing acrylamide from acrylonitrile using a biocatalyst having nitrile hydratase,
which comprises the step of keeping acrylonitrile while cooling to less than 30° C.

2. An apparatus for producing acrylamide from acrylonitrile using a biocatalyst having nitrile hydratase, which comprises a temperature regulation mechanism for maintaining the temperature of acrylonitrile at less than 30° C.


SEQUENCE LISTING (16 sequences)

SEQ ID NO: 1 - DNA, 690 bp, Rhodococcus rhodochrous
  CDS (1)..(690)
atggatggtatccacgacacaggcggcatgaccggatacggaccggtcccctatcagaaggacgagcccttcttccactacgagtgggagggtcggaccctgtcgattctgacctggatgcatctcaagggcatgtcgtggtgggacaagtcgcggttcttccgggagtcgatggggaacgaaaactacgtcaacgagattcgcaactcgtactacacccactggctgagtgcggcagaacgtatcctcgtcgccgacaagatcatcaccgaagaagagcgaaagcaccgtgtgcaggagatcctcgagggtcggtacacggacaggaacccgtcgcggaagttcgatccggccgagatcgagaaggcgatcgaacggcttcacgagccccactccctagcacttccaggagcggagccgagtttctccctcggtgacaaggtcaaagtgaagaatatgaacccgctgggacacacacggtgcccgaaatatgtgcggaacaagatcggggaaatcgtcacctcccacggctgccagatctatcccgagagcagctccgccggcctcggcgacgatccccgcccgctctacacggtcgcgttttccgcccaggaactgtggggcgacgacggaaacgggaaagacgtagtgtgcgtcgatctctgggaaccgtacctgatctctgcgtga

SEQ ID NO: 2 - protein, 229 aa, Rhodococcus rhodochrous
MDGIHDTGGMTGYGPVPYQKDEPFFHYEWEGRTLSILTWMHLKGMSWWDKSRFFRESMGNENYVNEIRNSYYTHWLSAAERILVADKIITEEERKHRVQEILEGRYTDRNPSRKFDPAEIEKAIERLHEPHSLALPGAEPSFSLGDKVKVKNMNPLGHTRCPKYVRNKIGEIVTSHGCQIYPESSSAGLGDDPRPLYTVAFSAQELWGDDGNGKDVVCVDLWEPYLISA

SEQ ID NO: 3 - DNA, 612 bp, Rhodococcus rhodochrous
  CDS (1)..(612)
gtgagcgagcacgtcaataagtacacggagtacgaggcacgtaccaaggcaatcgaaactttgctgtacgagcgagggctcatcacgcccgccgcggtcgaccgagtcgtttcgtactacgagaacgagatcggcccgatgggcggtgccaaggtcgtggcgaagtcctgggtggaccctgagtaccgcaagtggctcgaagaggacgcgacggccgcgatggcgtcattgggctatgccggtgagcaggcacaccaaatttcggcggtcttcaacgactcccaaacgcatcacgtggtggtgtgcactctgtgttcgtgctatccgtggccggtgcttggtctcccgcccgcctggtacaagagcatggagtaccggtcccgagtggtagcggaccctcgtggagtgctcaagcgcgatttcggtttcgacatccccgatgaggtggaggtcagggtttgggacagcagctccgaaatccgctacatcgtcatcccggaacggccggccggcaccgacggttggtccgaggacgagctggcgaagctggtgagtcgggactcgatgatcggtgtcagtaatgcgctcacaccccaggaagtgatcgtatga

SEQ ID NO: 4 - protein, 203 aa, Rhodococcus rhodochrous
VSEHVNKYTEYEARTKAIETLLYERGLITPAAVDRVVSYYENEIGPMGGAKVVAKSWVDPEYRKWLEEDATAAMASLGYAGEQAHQISAVFNDSQTHHVVVCTLCSCYPWPVLGLPPAWYKSMEYRSRVVADPRGVLKRDFGFDIPDEVEVRVWDSSSEIRYIVIPERPAGTDGWSEDELAKLVSRDSMIGVSNALTPQEVIV

SEQ ID NO: 5 - DNA, 315 bp, Rhodococcus rhodochrous
  CDS (1)..(315)
atgagtgaagacacactcactgatcggctcccggcgactgggaccgccgcaccgccccgcgacaatggcgagcttgtattcaccgagccttgggaagcaacggcattcggggtcgccatcgcgctttcggatcagaagtcgtacgaatgggagttcttccgacagcgtctcattcactccatcgctgaggccaacggttgcgaggcatactacgagagctggacaaaggcgctcgaggccagcgtggtcgactcggggctgatcagcgaagatgagatccgcgagcgcatggaatcgatggccatcatcgactga

SEQ ID NO: 6 - protein, 104 aa, Rhodococcus rhodochrous
MSEDTLTDRLPATGTAAPPRDNGELVFTEPWEATAFGVAIALSDQKSYEWEFFRQRLIHSIAEANGCEAYYESWTKALEASVVDSGLISEDEIRERMESMAIID

SEQ ID NO: 7 - DNA, 32 bp, Artificial
  Synthetic DNA
ggtctagaatggatggtatccacgacacaggc

SEQ ID NO: 8 - DNA, 34 bp, Artificial
  Synthetic DNA
cccctgcaggtcagtcgatgatggccatcgattc

SEQ ID NO: 9 - DNA, 702 bp, Psuedonocardia thermophila
  CDS (1)..(702)
atgaacggcgtgtacgacgtcggcggcaccgatgggctgggcccgatcaaccggcccgcggacgaaccggtcttccgcgccgagtgggagaaggtcgcgttcgcgatgttcccggcgacgttccgggccggcttcatgggcctggacgagttccggttcggcatcgagcagatgaacccggccgagtacctcgagtcgccgtactactggcactggatccgcacctacatccaccacggcgtccgcaccggcaagatcgatctcgaggagctggagcgccgcacgcagtactaccgggagaaccccgacgccccgctgcccgagcacgagcagaagccggagttgatcgagttcgtcaaccaggccgtctacggcgggctgcccgcaagccgggaggtcgaccgaccgcccaagttcaaggagggcgacgtggtgcggttctccaccgcgagcccgaagggccacgcccggcgcgcgcggtacgtgcgcggcaagaccgggacggtggtcaagcaccacggcgcgtacatctacccggacaccgccggcaacggcctgggcgagtgccccgagcacctctacaccgtccgcttcacggcccaggagctgtgggggccggaaggggacccgaactccagcgtctactacgactgctgggagccctacatcgagctcgtcgacacgaaggcggccgcggcatga

SEQ ID NO: 10 - protein, 233 aa, Psuedonocardia thermophila
MNGVYDVGGTDGLGPINRPADEPVFRAEWEKVAFAMFPATFRAGFMGLDEFRFGIEQMNPAEYLESPYYWHWIRTYIHHGVRTGKIDLEELERRTQYYRENPDAPLPEHEQKPELIEFVNQAVYGGLPASREVDRPPKFKEGDVVRFSTASPKGHARRARYVRGKTGTVVKHHGAYIYPDTAGNGLGECPEHLYTVRFTAQELWGPEGDPNSSVYYDCWEPYIELVDTKAAAA

SEQ ID NO: 11 - DNA, 618 bp, Psuedonocardia thermophila
  CDS (1)..(618)
atgaccgagaacatcctgcgcaagtcggacgaggagatccagaaggagatcacggcgcgggtcaaggccctggagtcgatgctcatcgaacagggcatcctcaccacgtcgatgatcgaccggatggccgagatctacgagaacgaggtcggcccgcacctcggcgcgaaggtcgtcgtgaaggcctggaccgacccggagttcaagaagcgtctgctcgccgacggcaccgaggcctgcaaggagctcggcatcggcggcctgcagggcgaggacatgatgtgggtggagaacaccgacgaggtccaccacgtcgtcgtgtgcacgctctgctcctgctacccgtggccggtgctggggctgccgccgaactggttcaaggagccgcagtaccgctcccgcgtggtgcgtgagccccggcagctgctcaaggaggagttcggcttcgaggtcccgccgagcaaggagatcaaggtctgggactccagctccgagatgcgcttcgtcgtcctcccgcagcgccccgcgggcaccgacgggtggagcgaggaggagctcgccaccctcgtcacccgcgagtcgatgatcggcgtcgaaccggcgaaggcggtcgcgtga

SEQ ID NO: 12 - protein, 205 aa, Psuedonocardia thermophila
MTENILRKSDEEIQKEITARVKALESMLIEQGILTTSMIDRMAEIYENEVGPHLGAKVVVKAWTDPEFKKRLLADGTEACKELGIGGLQGEDMMWVENTDEVHHVVVCTLCSCYPWPVLGLPPNWFKEPQYRSRVVREPRQLLKEEFGFEVPPSKEIKVWDSSSEMRFVVLPQRPAGTDGWSEEELATLVTRESMIGVEPAKAVA

SEQ ID NO: 13 - DNA, 435 bp, Psuedonocardia thermophila
  CDS (1)..(435)
gtgagcgccgaggcgaaggtccgcctgaagcactgccccacggccgaggaccgggcggcggccgacgcgctgctcgcgcagctgcccggcggcgaccgcgcgctcgaccgcggcttcgacgagccgtggcagctgcgggcgttcgcgctggcggtcgcggcgtgcagggcgggccggttcgagtggaagcagctgcagcaggcgctgatctcctcgatcggggagtgggagcgcacccacgatctcgacgatccgagctggtcctactacgagcacttcgtcgccgcgctggaatccgtgctcggcgaggaagggatcgtcgagccggaggcgctggacgagcgcaccgcggaggtcttggccaacccgccgaacaaggatcaccatggaccgcatctggagcccgtcgcggtccacccggccgtgcggtcctga

SEQ ID NO: 14 - protein, 144 aa, Psuedonocardia thermophila
VSAEAKVRLKHCPTAEDRAAADALLAQLPGGDRALDRGFDEPWQLRAFALAVAACRAGRFEWKQLQQALISSIGEWERTHDLDDPSWSYYEHFVAALESVLGEEGIVEPEALDERTAEVLANPPNKDHHGPHLEPVAVHPAVRS

SEQ ID NO: 15 - DNA, 32 bp, Artificial
  Synthetic DNA
ggtctagaatgaacggcgtgtacgacgtcggc

SEQ ID NO: 16 - DNA, 34 bp, Artificial
  Synthetic DNA
cccctgcaggtcaggaccgcacggccgggtggac